(19) United States
(12) Patent Application Publication
(10) Pub. No.: US 2011/0052735 A1
(43) Pub. Date: Mar. 3, 2011

(54) SHAMPOO WITH FRUIT JUICE AND/OR VEGETABLE JUICE

(75) Inventors: Erik Schulze Zur Wiesche, Hamburg (DE); Thomas Foerster, Duesseldorf (DE)

(73) Assignee: Henkel AG & Co. KGaA

(21) Appl. No.: 12/940,451

(22) Filed: Nov. 5, 2010

Related U.S. Application Data
(63) Continuation of application No. PCT/EP2009/051134, filed on Feb. 2, 2009.

(30) Foreign Application Priority Data
May 7, 2008 (DE) 10 2008 022 712.9

(51) Int. Cl. A61K 8/97; A61Q 1/00; A61Q 5/02; A61Q 5/12

(52) U.S. Cl. 424/732; 424/735; 424/736; 424/754; 424/757; 424/758; 424/765; 424/766; 424/777

(57) ABSTRACT

Shampoos providing hair treated therewith improved strength, handle and tensile strength, as well as combability in the dry and wet states and, at the same time, are particularly mild and ecologically compatible, and contain, in a cosmetically acceptable carrier, 0.1 to 35% by weight of at least one surfactant and 15 to 85% by weight of at least one fruit juice or vegetable juice.

### CROSS-REFERENCE TO RELATED APPLICATIONS

The present application is a continuation of International Patent Application No. PCT/EP2009/051134 filed 2 Feb. 2009, which claims priority to German Patent Application No. 10 2008 022 712.9 filed 7 May 2008, both of which are incorporated herein by reference.

The present invention relates to hair treatment agents containing fruit juice and/or vegetable juice or several such juices.

Regarding hair cleaning, increased hygiene consciousness is leading to more and more frequent cleaning of the hair. In order to be usable over the long term as well as provide optimum hair cleaning and care, agents used must be particularly mild to the hair and scalp. Although inherently mild surfactants from a wide variety of groups are known, they do not meet all consumer desires to a sufficient extent. It has been shown, for example, that frequent hair washing can negatively affect the structure of keratin fibers, necessitating the addition of specific substances that strengthen the internal structure of the keratin fibers, thereby improving the strength, softness, and tensile strength of keratin fibers. Accordingly, there is a need for active substances or active-substance combinations for hair cleaning agents that not only do not impair, but rather improve the structure of fibers treated with them. Further, there is increasing consumer demand to use LOHAS-compatible cosmetics. LOHAS is a marketing term that stands for “Lifestyle of Health and Sustainability,” and defines a new type of lifestyle and consumer who is oriented in his or her consumption behavior toward health and sustainability. The present invention provides hair cleaning agents that impart to hair treated with these agents advantageous properties with respect to strength, softness, and tensile strength as well as combability in the dry and wet state, and that are particularly mild and environmentally compatible.

It is known to use plant extracts in cosmetic products in order to give the products a specific properties profile. It has now been found that large quantities of fruit juices and/or vegetable juices have very advantageous effects on keratin fibers. This effect can be further enhanced by a specific formulation of the products, for example, by formulating the products in PEG-free fashion.

The subject matter of the present application is, in a first embodiment, hair treatment agents containing, in a cosmetically acceptable carrier, based on their weight—
(a) 0.1 to 35 wt % of at least one surfactant, and
(b) 15 to 85 wt % of at least one fruit juice or vegetable juice.

Agents according to the present invention can be formulated, for example, as hair shampoos, hair conditioners, conditioning shampoos, hair sprays, hair rinses, hair therapies, hair packings, hair tonics, permanent-wave fixing solutions, hair coloring shampoos, hair coloring agents, hair setting lotions, hair setting agents, hair styling preparations, blow-dry lotions, foam setting agents, hair gels, hair waxes, and other hair cleaning and hair treatment agents. They can be formulated as products that remain on the hair (“leave-on”) or that are washed off (“rinse-off”), the latter being preferred.

Preferred agents contain at least one fruit juice, the juice preferably being used within narrower quantitative ranges. Preferred hair treatment agents contain, based on their weight, 20 to 80 wt %, preferably 25 to 75 wt %, more preferably 30 to 70 wt %, and particularly 35 to 60 wt % of at least one fruit juice.

For purposes of the present invention, “fruit juice” is the fermentable, but not fermented, juice obtained by means of mechanical methods from fruits. These fruit juices include juices (called re-diluted fruit juices) reconstituted from concentrated fruit juice (called re-diluted fruit juices) by adding water (in the form of water of suitable quality) removed upon concentration and volatile flavor compounds that were separated out. The fruit flesh removed upon concentration of the original fruit juice or juices of the same fruit species can be added to the reconstituted fruit juice up to the quantity contained in the original juice. Fruit juices are produced and packaged either directly from fresh fruit as “direct juice” or from semi-finished products such as fruit juice concentrates or from powder-containing fruit pulp or fruit pulp concentrates.

Particularly preferred fruit juices for use include (individually or mixed with one another) apple juice and/or orange juice and/or other citrus fruits. Hair treatment agents containing at least one fruit juice chosen from apple juice, orange juice, grape juice, lemon juice, lime juice, sweet lime juice, grapefruit juice, blackcurrant juice, cherry juice, morello cherry juice, apricot juice, peach juice, nectarine juice, damson juice, yellow plum juice, passion fruit juice, plum juice, greengage juice, blackberry juice, raspberry juice, strawberry juice, pineapple juice, pomegranate juice, lychee juice, mango juice, tangerine juice, papaya juice, pitaya juice, carambola juice, pear juice, quince juice, loquat juice, serviceberry juice, rowan berry juice, gooseberry juice, blueberry juice, foxberry juice, tayberry juice, cranberry juice, elderberry juice, chokeberry juice, sea buckthorn berry juice, are preferred.

In addition to or instead of fruit juice or juices, agents according to the present invention can contain one or more vegetable juices. Here also, quantities used preferably fall within narrow ranges. Preferred hair treatment agents contain, based on their weight, 20 to 80 wt %, preferably 25 to 75 wt %, more preferably 30 to 70 wt %, and particularly 35 to 60 wt % of at least one vegetable juice. “Vegetable juice” refers to undiluted, preserved juice from a vegetable after comminution, pressing or centrifuging, and pasteurization or sterilization. According to German food code, vegetable juice is “the undiluted, liquid, fermentable and unfermented or lacto-fermented product from a vegetable, intended for direct consumption”. In this regard, vegetable juice can also be produced as a concentrate from vegetable juice or vegetable pulp.

Analogously to fruit juice, vegetable juice concentrate or vegetable pulp concentrate can be produced by concentration, thereby reducing storage and transportation costs. To produce the juice, the quantity of water removed is then added back in. (The water must be at least of drinking-water quality.) If applicable, the flavor of the vegetable juice is reconstituted with the aid of the volatile flavor compounds captured when the vegetable juice was concentrated. Temporary concentration should be apparent in the juice description, for example, by the statement “from . . . concentrate”. Tomatoes are typically processed, but cucumbers, carrots, radishes, beets, sauerkraut, celery, and spinach can be processed as well. In contrast to fruit juice, cellulolytic and hemicellulolytic enzymes may also be used in vegetable juice production. Acidification using lactic acid fermentation or added citric acid is usual. Particularly preferred vegetable juices include rhubarb juice and/or Swiss chard juice, individually or mixed with one another. Hair treatment agents containing at least one vegetable juice chosen from rhubarb juice, kohlrabi juice, celeriac juice, carrot juice, beet juice, onion juice, leek juice, garlic juice, cabbage juice, asparagus juice, spinach juice, Swiss chard juice, tomato juice, cucumber juice, pumpkin juice, pea juice, bean juice, artichoke juice, cauliflower juice, kale juice, red cabbage juice, broccoli juice, and/or Brussels sprout juice, are preferred.

For environmental reasons and in consideration of sustainability aspects, fruits and vegetables grown organically are preferred. Hair treatment agents according to the present invention that contain exclusively fruit juices and/or vegetable juices grown under monitored organic conditions are therefore particularly preferred.

It has further been found that agents according to the invention are preferably formulated free of polyethylene glycols, as polyethylene glycols can detract from the positive effects of the fruit juices and/or vegetable juices. Preferred agents according to the present invention therefore contain no compounds of the following formula (I)—

H—(O—CH₂—CH₂)ₙ—OH (I)

wherein n is a whole number from 1 to 100,000.

Hair treatment agents free of polyethylene glycols of general formula (I) contain neither ethylene glycol (n=1) nor products having a degree of polymerization Pₙ=2 to 4 (e.g., diethylene glycol, triethylene glycol, and tetraethylene glycol), nor polyethylene glycols having higher degrees of polymerization Pₙ, from approx. 5 to 100,000, which can no longer be manufactured with molar uniformity but instead are polydisperse.

Surprisingly, by omitting compounds of formula (I), the fruit juices and/or vegetable juices can exert their effect on hair much better. Strengthening of keratinic fibers, expressed as improved combability in the dry and particularly in the wet state, as well as fullness and suppleness, are particularly improved.

Agents according to the present invention are preferably free not only of polyethylene glycols but also of polypropylene glycols (PPG). Preferred hair treatment agents contain no compounds according to the following formula (II)—

H—(O—CH(CH₃)CH₂)ₙ—OH (II)

wherein n is a whole number from 1 to 100,000.

Hair treatment agents free of polypropylene glycols of the general formula (II) contain neither propylene glycol (n=1) nor products having a degree of polymerization Pₙ=2 to 4 (dipropylene glycol, tripropylene glycol, and tetrapropylene glycol), nor polypropylene glycols having higher degrees of polymerization Pₙ from approx. 5 to 100,000, which can no longer be manufactured with molar uniformity but instead are polydisperse. These compounds, too, prevent fruit juices and/or vegetable juices from exerting their effect on hair, so that improved effects can be observed by omitting their use.

It has been found that use of ethoxylated compounds should preferably also be avoided. In particular, nonionic surfactants of the alkyl ethoxylate or alkenyl ethoxylate types likewise decrease the positive effects of the fruit juices and/or vegetable juices, and therefore should not be used in the formulations. Further preferred hair cleaning agents therefore contain no compounds according to the following formula (III)—

R—(O—CH₂CH₂)ₙ—OH (III)

wherein R is any alkyl residue or alkenyl residue and n is a whole number from 1 to 100,000.

Ethoxylated anionic surfactants can also, although to a lesser extent, detract from the positive effect of fruit juices and/or vegetable juices. Alkyl ether sulfates have proven especially problematic here, while alkyl ether carboxylates exhibit less of an effect-diminishing action than ether sulfates. Preferred hair treatment agents according to the present invention therefore contain no compounds according to the following formula (IV)—

R—(O—CH₂CH₂)ₙ—OSO₃H (IV)

wherein R is any alkyl residue or alkenyl residue and n is a whole number from 1 to 100,000.

In particularly preferred agents according to the present invention, use of compounds containing ethoxylated groupings is entirely dispensed with. Interestingly, a hydroxyethyl grouping (e.g., in certain surfactants) does not impair the effect of fruit juices and/or vegetable juices, while ethylene glycol does. Preferred hair treatment agents therefore contain no compound having the —(O—CH₂CH₂)ₙ—O— group, where n is 1 to 10,000. Particularly preferred hair treatment agents contain no compounds having the grouping of formula (V)—

—(O—CH₂—CH₂)ₙ— (V)

wherein n is a whole number from 2 to 100,000.

Agents according to the present invention contain 0.1 to 35 wt % of at least one surfactant. As already mentioned above, ethoxylated nonionic surfactants are preferably excluded, and anionic surfactants from the groups of the alkyl ether sulfates are also preferably not used. As will be set forth in detail below, preferred agents are likewise free of alkyl sulfates. Use of amphoteric and/or zwitterionic surfactants is particularly preferred.

Particularly preferred hair treatment agents contain amphoteric surfactant(s) chosen from N-alkylglycines, N-alkylpropionic acids, N-alkylaminobutyric acids, N-alkyliminodipropionic acids, N-hydroxyethyl-N-alkylamidopropylglycines, N-alkyltaurines, N-alkylsarcosines, 2-alkylaminopropionic acids having approximately 8 to 24 carbon atoms in the alkyl group, alkylaminoacetic acids having approximately 8 to 24 carbon atoms in the alkyl group, N-cocoalkylaminopropionate, cocoacylaminoethylaminopropionate, C₁₂- to C₁₋₈-acyl sarcosine, N-alkyl-N,N-dimethylammonium glycinates (e.g., cocoalkyldimethylammonium glycinate), N-acylaminopropyl-N,N-dimethylammonium glycinates (e.g., cocoacylaminopropyldimethylammonium glycinate), 2-alkyl-3-carboxymethyl-3-hydroxyethylimidazolines having 8 to 18 carbon atoms in the alkyl or acyl group, cocoacylaminoethylhydroxyethylcarboxymethyl glycinate, compounds known by the INCI name Cocoamidopropyl Betaine, and compounds known by the INCI name Disodium Cocoamphodiacetate. Preferred agents contain the amphoteric surfactant(s) in quantities from 0.5 to 9 wt %, preferably from 0.75 to 8 wt %, and particularly from 1 to 7.5 wt %, based on total weight of the agent.

Particularly preferred hair treatment agents contain as amphoteric surfactants betaines according to the following formula (VI)—

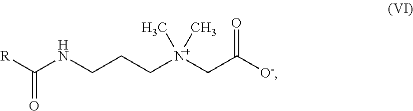

wherein R is a straight-chain or branched, saturated or mono- or polyunsaturated alkyl or alkenyl residue having 8 to 24 carbon atoms. These surfactants are referred to in INCI nomenclature as Amidopropyl Betaines, with representatives derived from coconut fatty acids being preferred and being referred to as Cocoamidopropyl Betaines. It is particularly preferred to use surfactants of formula (VI) that are a mixture of the following constituents: H₃C—(CH₂)₇—C(O)—NH—(CH₂)₃N⁺(CH₃)₂CH₂COO⁻, H₃C—(CH₂)₉—C(O)—NH—(CH₂)₃N⁺(CH₃)₂CH₂COO⁻, H₃C—(CH₂)₁₁—C(O)—NH—(CH₂)₃N⁺(CH₃)₂CH₂COO⁻, H₃C—(CH₂)₁₃—C(O)—NH—(CH₂)₃N⁺(CH₃)₂CH₂COO⁻, H₃C—(CH₂)₁₅—C(O)—NH—(CH₂)₃N⁺(CH₃)₂CH₂COO⁻, and H₃C—(CH₂)₇—CH═CH—(CH₂)₇—C(O)—NH—(CH₂)₃N⁺(CH₃)₂CH₂COO⁻.

Preferably, surfactants of formula (VI) are used within narrower quantitative ranges. Preferred agents in this context contain, based on their weight, 0.25 to 8 wt %, more preferably 0.5 to 7 wt %, more preferably 0.75 to 6.5 wt %, and particularly 1 to 5.5 wt % surfactants of formula (VI).

In addition to or instead of amphoteric surfactant or surfactants of formula (VI), hair treatment agents according to the invention can contain as amphoteric surfactants betaines according to the following formula (VII)—

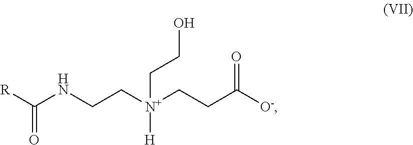

wherein R is a straight-chain or branched, saturated or mono- or polyunsaturated alkyl or alkenyl residue having 8 to 24 carbon atoms. These surfactants are referred to in INCI nomenclature as Amphoacetates, with representatives derived from coconut fatty acids being preferred and being referred to as Cocoamphoacetates. For reasons of production engineering, surfactants of this type also contain betaines of formula (VIIa)—

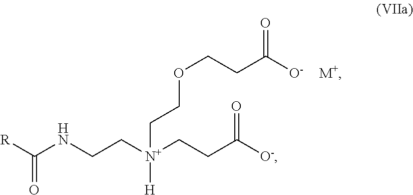

wherein R is a straight-chain or branched, saturated or mono- or polyunsaturated alkyl or alkenyl residue having 8 to 24 carbon atoms and M denotes a cation. These surfactants are referred to in INCI nomenclature as Amphodiacetates, with representatives derived from coconut fatty acids being preferred and being referred to as Cocoamphodiacetates. It is particularly preferred to use surfactants of formula (VII) that are a mixture of the following constituents:

H₃C—(CH₂)₇—C(O)—NH—(CH₂)₂NH⁺(CH₂CH₂OH)CH₂CH₂COO⁻,

H₃C—(CH₂)₉—C(O)—NH—(CH₂)₂NH⁺(CH₂CH₂OH)CH₂CH₂COO⁻,

H₃C—(CH₂)₁₁—C(O)—NH—(CH₂)₂NH⁺(CH₂CH₂OH)CH₂CH₂COO⁻,

H₃C—(CH₂)₁₃—C(O)—NH—(CH₂)₂NH⁺(CH₂CH₂OH)CH₂CH₂COO⁻,

H₃C—(CH₂)₁₅—C(O)—NH—(CH₂)₂NH⁺(CH₂CH₂OH)CH₂CH₂COO⁻, and

H₃C—(CH₂)₇—CH═CH—(CH₂)₇—C(O)—NH—(CH₂)₂NH⁺(CH₂CH₂OH)CH₂CH₂COO⁻.

Preferably, surfactants of formula (VII) are used within narrower quantitative ranges. Preferred agents in this context contain, based on their weight, 0.25 to 8 wt %, more preferably 0.5 to 7 wt %, more preferably 0.75 to 6.5 wt %, and particularly 1 to 5.5 wt % surfactants of formula (VII). In summary, hair treatment agents wherein the residue R in formulae (VI) and (VII) is chosen from H₃C—(CH₂)₇—, H₃C—(CH₂)₉—, H₃C—(CH₂)₁₁—, H₃C—(CH₂)₁₃—, H₃C—(CH₂)₁₅—, H₃C—(CH₂)₇—CH═CH—(CH₂)₇—, or mixtures thereof, are preferred.

Alkylpolyglycosides (APG) are particularly preferred nonionic surfactants. Hair treatment agents containing, based on their weight, as nonionic surfactants 0.1 to 20 wt % alkylpolyglycosides of the general formula RO—(Z)ₓ, where R is alkyl, Z sugar, and x the number of sugar units, are accordingly preferred.

Agents according to the present invention can contain the alkyl and/or alkenyl oligoglucosides and acyl aspartates likewise at a weight ratio of 1:99 to 99:1, preferably 10:90 to 90:10, and particularly 80:20 to 50:50.

The care-providing effect of agents according to the present invention can be further intensified by using specific care-providing substances. These are preferably chosen from specific groups of care-providing substances known per se, since these care-providing substances harmonize outstandingly, in terms of formulation engineering and care-providing effect, with fruit juices and/or vegetable juices used according to the present invention.

Preferred hair treatment agents can additionally contain, based on their weight, (a) care-providing substance(s) in amounts of from 0.001 to 10 wt %, preferably 0.005 to 7.5 wt %, particularly preferably 0.01 to 5 wt %, and particularly 0.05 to 2.5 wt %, with the care-providing substance(s) preferably being chosen from—

i) L-carnitine and/or salts thereof;

ii) panthenol and/or pantothenic acid;

iii) 2-furanones and/or derivatives thereof, in particular pantolactone;

iv) taurine and/or salts thereof;

v) niacinamide;

vi) ubiquinone;

vii) ectoin; and

viii) allantoin.

Accordingly, hair treatment agents of this embodiment combine the surfactant and fruit juice and/or vegetable juice with at least one care-providing substance chosen from L-carnitine and/or salts thereof, panthenol and/or pantothenic acid, 2-furanones and/or derivatives thereof, in particular pantolactone, taurine and/or salts thereof, niacinamide, ubiquinone, ectoin, allantoin. These care-providing substances are described in the priority document.

Preferred hair treatment agents contain, based on their weight, 0.001 to 10 wt %, preferably 0.005 to 7.5 wt %, particularly preferably 0.01 to 5 wt %, and in particular 0.05 to 2.5 wt % L-carnitine or L-carnitine derivatives, preferred L-carnitine derivatives being chosen from acetyl-L-carnitine, L-carnitine fumarate, L-carnitine citrate, lauroyl-L-carnitine, and particularly L-carnitine tartrate.

Preferred hair treatment agents can contain, based on their weight, 0.01 to 5 wt %, by preference 0.05 to 2.5 wt %, particularly preferably 0.1 to 1.5 wt %, and in particular 0.25 to 1 wt % panthenol ((±)-2,4-dihydroxy-N-(3-hydroxypropyl)-3,3-dimethylbutyramide).

Preferred hair treatment agents can contain, based on their weight, 0.01 to 15 wt %, preferably 0.025 to 12.5 wt %, particularly preferably 0.05 to 10 wt %, more preferably 0.1 to 7.5 wt %, and in particular 0.5 to 5 wt % of at least one 2-furanone derivative of formula (Fur-I) and/or formula (Fur-II)—

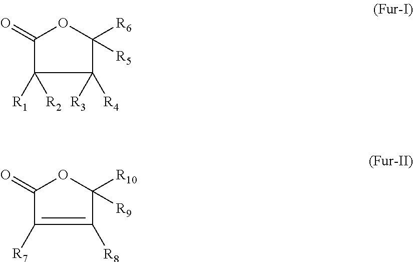

wherein the residues R¹ to R¹⁰ mutually independently are:
hydrogen, —OH, a methyl, methoxy, aminomethyl, or hydroxymethyl residue;
a C₂ to C₄ saturated or mono- or diunsaturated, branched or linear hydrocarbon residue;
a C₂ to C₄ saturated or mono- or diunsaturated, branched or linear mono-, di- or trihydroxy hydrocarbon residue;
a C₂ to C₄ saturated or mono- or diunsaturated, branched or linear mono-, di- or triamino hydrocarbon residue;
an —OR¹¹ group, wherein R¹¹ is a C₂ to C₄ saturated or mono- or diunsaturated, branched or linear hydrocarbon residue, a C₂ to C₄ saturated or mono- or diunsaturated, branched or linear mono-, di- or trihydroxy hydrocarbon residue;
an —NR¹²R¹³ group, wherein R¹² and R¹³ each mutually independently are hydrogen, a methyl residue, a C₂ to C₄ saturated or mono- or diunsaturated, branched or linear hydrocarbon residue, a C₂ to C₄ saturated or mono- or diunsaturated, branched or linear mono-, di- or trihydroxy hydrocarbon residue;
a —COOR¹⁴ group, wherein R¹⁴ is hydrogen, a methyl residue, a C₂ to C₄ saturated or mono- or diunsaturated, branched or linear hydrocarbon residue, a C₂ to C₄ saturated or mono- or diunsaturated, branched or linear mono-, di- or trihydroxy hydrocarbon residue, a C₂ to C₄ saturated or mono- or diunsaturated, branched or linear mono-, di- or triamino hydrocarbon residue;
a —CONR¹⁵R¹⁶ group, wherein R¹⁵ and R¹⁶ each mutually independently are hydrogen, methyl, a C₂ to C₄ saturated or mono- or diunsaturated, branched or linear hydrocarbon residue, a C₂ to C₄ saturated or mono- or diunsaturated, branched or linear mono-, di- or trihydroxy hydrocarbon residue, a C₂ to C₄ saturated or mono- or diunsaturated, branched or linear mono-, di- or triamino hydrocarbon residue;
a —COR¹⁶ group, wherein R¹⁶ is a methyl residue, a C₂ to C₄ saturated or mono- or diunsaturated, branched or linear hydrocarbon residue, a C₂ to C₄ saturated or mono- or diunsaturated, branched or linear mono-, di- or trihydroxy hydrocarbon residue, a C₂ to C₄ saturated or mono- or diunsaturated, branched or linear mono-, di- or triamino hydrocarbon residue; or
an —OCOR¹⁷ group, wherein R¹⁷ is a methyl residue, a C₂ to C₃₀ saturated or mono- or polyunsaturated, branched or linear hydrocarbon residue, a C₂ to C₃₀ saturated or mono- or polyunsaturated, branched or linear mono-, di-, tri-, or polyhydroxy hydrocarbon residue, a C₂ to C₃₀ saturated or mono- or polyunsaturated, branched or linear mono, di-, tri-, or polyamino hydrocarbon residue;
   with the proviso that when R⁷ and R⁸ are —OH and R⁹ or R¹⁰ are simultaneously hydrogen, the remaining group R⁹ or R¹⁰ is not a dihydroxylethyl residue

Preferred hair treatment agents contain, based on their weight, 0.01 to 15 wt %, by preference 0.025 to 12.5 wt %, particularly preferably 0.05 to 10 wt %, more preferably 0.1 to 7.5 wt %, and in particular 0.5 to 5 wt % taurine (2-aminoethanesulfonic acid).

In summary, preferred hair treatment agents contain, based on their weight, 0.1 to 5 wt %, preferably 0.2 to 4 wt %, particularly preferably 0.25 to 3.5 wt %, more preferably 0.5 to 3 wt %, and in particular 0.5 to 2.5 wt % vitamins and/or provitamins and/or vitamin precursors that by preference are assigned to the groups A, B, C, E, F, and H, such that preferred agents contain 2,4-dihydroxy-N-(3-hydroxypropyl)-3,3-dimethylbutyramide (provitamin B₅) and/or pantothenic acid (vitamin B₃, vitamin B₅) and/or niacin, niacinamide or nicotinamide (vitamin B₃) and/or L-ascorbic acid (vitamin C) and/or thiamin (vitamin B₁) and/or riboflavin (vitamin B₂, vitamin G) and/or biotin (vitamin B₇, vitamin H) and/or folic acid (vitamin B₉, vitamin B_{c} or vitamin M) and/or vitamin B₆ and/or vitamin B₁₂.

It has been found that specific quinones possess a particular suitability as a care-providing substance. Agents according to the present invention can therefore contain as a further care-providing substance 0.0001 to 5 wt % of at least one bioquinone of formula (Ubi)—

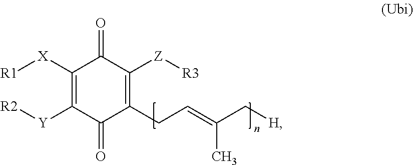

wherein
X, Y, Z mutually independently are —O— or —NH— or —NR⁴— or a chemical bond;
R¹, R², R³ mutually independently are a hydrogen atom or an optionally substituted aryl group or an optionally substituted (C₁ to C₆) alkyl group or a hydroxyalkyl group or a polyhydroxyalkyl group or an optionally substituted (C₁ to C₆) alkylene group, or a (C₁ to C₆) acyl group, preferred residues being chosen mutually independently from —H, —CH₃, —CH₂CH₃, —(CH₂)₂CH₂, —CH(CH₃)₂, —(CH₂)₃CH₃, —CH(CH₃)CH₂CH₃, —CH₂CH(CH₃)₂, —C(CH₃)₃;
R⁴ is —CH₃, —CH₂CH₃, —(CH₂)₂ CH₂, —CH(CH₃)₂, —(CH₂)₃ CH₃, —CH(CH₃)CH₂CH₃, —CH₂CH(CH₃)₂, —C(CH₃)₃; and
n is a value from 1 to 20, by preference from 2 to 15, and in particular 5, 6, 7, 8, 9, 10.

Particularly preferred hair treatment agents contain as a care-providing substance, based on their weight, 0.0001 to 1 wt %, preferably 0.001 to 0.5 wt %, and particularly preferably 0.005 to 0.1 wt % of at least one ubiquinone and/or at least one ubiquinol and/or at least one derivative of said substances, such that preferred agents contain a ubiquinone of formula (Ubi)—

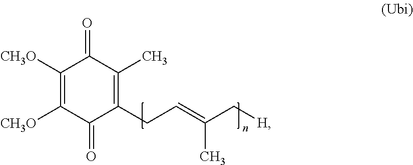

wherein n is 6, 7, 8, 9, or 10, particularly preferably 10 (Coenzyme Q10).

Alternatively or in addition to the particularly preferred ubiquinones, agents according to the present invention can also contain plastoquinones. Preferred agents in this context contain 0.0002 to 4 wt %, by preference 0.0005 to 3 wt %, particularly preferably 0.001 to 2 wt %, more preferably 0.0015 to 1, and in particular 0.002 to 0.5 wt % of at least one plastoquinone of formula (Ubi-b)—

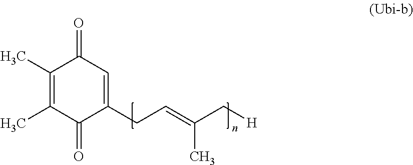

wherein n is a value from 1 to 20, preferably from 2 to 15, and in particular 5, 6, 7, 8, 9, 10, such that particularly preferred agents contain plastoquinone PQ-9 of the formula—

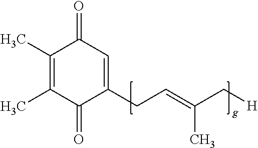

Preferred hair treatment agents can contain, based on their weight, 0.001 to 10 wt %, by preference 0.01 to 5 wt %, particularly preferably 0.05 to 2.5 wt %, and in particular 0.1 to 1 wt % (S)-2-methyl-1,4,5,6-tetrahydro-4-pyrimidinecarboxylic acid (ectoin) as well as the physiologically acceptable salts of said compound, and/or (S,S)-5-hydroxy-2-methyl-1,4,5,6-tetrahydro-4-pyrimidinecarboxylic acid (hydroxyectoin) as well as the physiologically acceptable salts of said compound.

Hair treatment agents according to the present invention that are particularly preferred contain, based on their weight, 0.001 to 10 wt %, by preference 0.01 to 5 wt %, particularly preferably 0.05 to 2.5 wt %, and in particular 0.1 to 1 wt % 5-ureidohydantoin (allantoin).

Preferred agents according to the present invention contain purine and/or purine derivatives within narrower quantity ranges. In this context, cosmetic agents preferred according to the present invention are characterized in that in that they contain, based on their weight, 0.001 to 2.5 wt %, by preference 0.0025 to 1 wt %, particularly preferably 0.005 to 0.5 wt %, and in particular 0.01 to 0.1 wt % purine(s) and/or purine derivative(s).

Some representatives of purine(s) and purine derivatives are particularly preferred according to the present invention. Such preferred hair treatment agents contain as a care-providing substance, based on their weight, 0.001 to 2.5 wt %, preferably 0.0025 to 1 wt %, particularly preferably 0.005 to 0.5 wt %, and in particular 0.01 to 0.1 wt % purine(s) and/or purine derivative(s), such that preferred agents contain purine and/or purine derivative(s) of formula (Pur-I)—

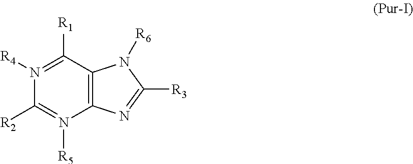

wherein the residues R¹, R², and R³ are chosen mutually independently from —H, —OH, NH₂, —SH, and the residues R⁴, R⁵, and R⁶ are chosen mutually independently from —H, —CH₃, and —CH₂—CH₃, with the following compounds being preferred: purine (R¹═R²═R³═R⁴═R⁵ ═R⁶ ═H), adenine (R¹═NH₂, R²═R³ ═R⁴ ═R⁵ ═R⁶ ═H), guanine (R¹═OH, R²═NH₂, R³═R⁴═R⁵═R⁶═H), uric acid (R¹═R²═R³═OH, R⁴═R⁵═R⁶═H), hypoxanthine (R¹═OH, R²═R³═R⁴═R⁵═R⁶═H), 6-purinethiol (R¹═SH, R² ═R³ ═R⁴ ═R⁵ ═R⁶ ═H), 6-thioguanine (R¹═SH, R²═NH₂, R³═R⁴ ═R⁵═R⁶ ═H), xanthine (R¹═R²═OH, R³ ═R⁴═R⁵ ═R⁶ ═H), caffeine (R¹═R²═OH, R³ ═H, R⁴═R⁵ ═R⁶ ═CH₃), theobromine (R¹═R²═OH, R³═R⁴═H, R⁵═R⁶═CH₃), theophylline (R¹═R²═OH, R³═H, R⁴═CH₃, R⁵═CH₃, R⁶═H).

It is further advantageous to use purine or purine derivatives and bioquinones at a specific ratio to one another. Accordingly, preferred agents in this context have a weight ratio of purine (derivative(s)) to bioquinone(s) equal to 10:1 to 1:100, preferably 5:1 to 1:50, particularly preferably 2:1 to 1:20, and in particular 1:1 to 1:10.

As already mentioned, caffeine is a particularly preferred purine derivative and Coenzyme Q10 is a particularly preferred bioquinone. Particularly preferred agents according to the invention therefore can contain, based on their weight, 0.001 to 2.5 wt %, preferably 0.0025 to 1 wt %, particularly preferably 0.005 to 0.5 wt %, and in particular 0.01 to 0.1 wt % caffeine, and 0.0002 to 4 wt %, by preference 0.0005 to 3 wt %, particularly preferably 0.001 to 2 wt %, more preferably 0.0015 to 1, and in particular 0.002 to 0.5 wt % Coenzyme Q10.

Particularly preferred hair treatment agents can contain, based on their weight, 0.001 to 2.5 wt %, by preference 0.0025 to 1 wt %, particularly preferably 0.005 to 0.5 wt %, and in particular 0.01 to 0.1 wt % flavonoids, in particular flavonols, particularly preferably 3,3′,4′,5,7-pentahydroxyflavone (quercetin) and/or 3,3′,4′,5,7-pentahydroxyflavone-3-O-rutinoside (rutin).

Use of bisabolol and/or bisabolol oxides as a care-providing substance in the agents is also preferred. Preferred in this context are hair treatment agents additionally containing 0.001 to 5 wt %, preferably 0.01 to 4 wt %, particularly preferably 0.02 to 2.5 wt %, and in particular 0.1 to 1.5 wt % bisabolol and/or oxides of bisabolol, by preference (−)-alpha-bisabolol.

Creatine is also suitable according to the present invention as a care-providing substance. Particularly preferred hair treatment agents contain, based on their weight, 0.01 to 15 wt %, preferably 0.025 to 12.5 wt %, particularly preferably 0.05 to 10 wt %, more preferably 0.1 to 7.5 wt %, and in particular 0.5 to 5 wt % N-methylguanidoacetic acid (creatine).

Agents according to the present invention can contain, in addition to the ingredients mentioned above and optional further ingredients, further substances that prevent, mitigate, or cure hair loss. A content of hair-root-stabilizing active substances is particularly advantageous. In summary, hair treatment agents according to the present invention that additionally contain, based on their weight, 0.001 to 5 wt % hair-root-stabilizing substances, in particular minoxidil and/or finasteride and/or ketoconazol, are preferred.

Hair treatment agents additionally containing, based on their weight, 0.001 to 5 wt % anti-dandruff active substances, in particular piroctone olamine (1-hydroxy-4-methyl-6-(2,4,4-trimethylpentyl)pyridin-2(1H)-one, compound with 2-aminoethanol 1:1) and/or zinc pyrithione and/or selenium sulfide and/or climbazole and/or salicylic acid or fumaric acid, are preferred.

In addition to the above care-providing substances, agents according to the invention can contain further care-providing substances. Their presence is not absolutely necessary in order to achieve the effects according to the present invention, but additional effects, such as a pleasant feel or pleasant application haptics, can result from the use of these care-providing substances. Agents according to the invention can, with particular preference, contain one or more amino acids as a further ingredient. Useful amino acids include glycine, alanine, valine, leucine, isoleucine, phenylalanine, tyrosine, tryptophan, proline, aspartic acid, glutamic acid, asparagine, glutamine, serine, threonine, cysteine, methionine, lysine, arginine, histidine, (β-alanine, 4-aminobutyric acid (GABA), betaine, L-cystine (L-CySS), L-carnitine, L-citrulline, L-theanine, 3′,4′-dihydroxy-L-phenylalanine (L-DOPA), 5′-hydroxy-L-tryptophan, L-homocysteine, S-methyl-L-methionine, S-allyl-L-cysteine sulfoxide (L-alliine), L-trans-4-hydroxyproline, L-5-oxoproline (L-pyroglutamic acid), L-phosphoserine, creatine, 3-methyl-L-histidine, L-ornithine; both the individual amino acids and mixtures can be used. Preferred agents contain one or more amino acids in narrower quantity ranges. Preferred hair treatment agents contain as a care-providing substance, based on their weight, 0.01 to 5 wt %, by preference 0.02 to 2.5 wt %, particularly preferably 0.05 to 1.5 wt %, more preferably 0.075 to 1 wt %, and in particular 0.1 to 0.25 wt % amino acid(s), preferably from glycine and/or alanine and/or valine and/or lysine and/or leucine and/or threonine.

As a further constituent, agents according to the invention can contain at least one carbohydrate chosen from monosaccharides, disaccharides, and/or oligosaccharides. Hair treatment agent here contain as a care-providing substance, based on their weight, 0.01 to 5 wt %, preferably 0.05 to 4.5 wt %, particularly preferably 0.1 to 4 wt %, more preferably 0.5 to 3.5 wt %, and in particular 0.75 to 2.5 wt % carbohydrate(s) chosen from monosaccharides, disaccharides, and/or oligosaccharides, with preferred carbohydrates chosen from monosaccharides, in particular, D-ribose and/or, D-xylose and/or, L-arabinose and/or, D-glucose and/or, D-mannose and/or, D-galactose and/or, D-fructose and/or, sorbose and/or, L-fucose and/or, L-rhamnose, disaccharides, in particular, sucrose and/or, maltose and/or, lactose and/or, trehalose and/or, cellobiose and/or, gentobiose and/or isomaltose.

Particularly preferred agents according to the present invention contain, based on their weight —

0.005 to 0.015 wt % caffeine and 0.75 to 1.5 wt % glucose monohydrate;

0.005 to 0.015 wt % caffeine and 0.75 to 1.5 wt % sucrose;

0.005 to 0.015 wt % caffeine and 0.75 to 1.5 wt % fructose.

Particularly preferred agents according to the present invention contain, based on their weight—
0.005 to 0.015 wt % caffeine+0.75 to 1.5 wt % glucose monohydrate+0.1 to 0.25 wt % glycine,
0.005 to 0.015 wt % caffeine+0.75 to 1.5 wt % sucrose+0.1 to 0.25 wt % glycine,
0.005 to 0.015 wt % caffeine+0.75 to 1.5 wt % fructose+0.1 to 0.25 wt % glycine,
0.005 to 0.015 wt % caffeine+0.75 to 1.5 wt % glucose monohydrate+0.1 to 0.25 wt % alanine,
0.005 to 0.015 wt % caffeine+0.75 to 1.5 wt % sucrose+0.1 to 0.25 wt % alanine,
0.005 to 0.015 wt % caffeine+0.75 to 1.5 wt % fructose+0.1 to 0.25 wt % alanine,
0.005 to 0.015 wt % caffeine+0.75 to 1.5 wt % glucose monohydrate+0.1 to 0.25 wt % valine,
0.005 to 0.015 wt % caffeine+0.75 to 1.5 wt % sucrose+0.1 to 0.25 wt % valine,
0.005 to 0.015 wt % caffeine+0.75 to 1.5 wt % fructose+0.1 to 0.25 wt % valine.

A particularly preferred group of ingredients is silicones. Particularly preferred agents contain silicone(s) in amounts of from 0.1 to 10 wt %, preferably from 0.25 to 7 wt %, and particularly from 0.5 to 5 wt %, based on total weight of the agent. Preferred silicones are described below. Particularly preferred agents contain at least one silicone of formula (Si-I)—

(CH₃)₃Si—[O—Si(CH₃)₂]ₓ—O—Si(CH₃)₃ (Si-I)

wherein x is a number from 0 to 100, preferably from 0 to 50, more preferably from 0 to 20, and particularly 0 to 10. Preferred silicones usable according to the invention have viscosities from 0.2 to 2 mm²s⁻¹ at 20° C., with silicones having viscosities from 0.5 to 1 mm²s⁻¹ being particularly preferred.

Particularly preferred agents contain one or more aminofunctional silicones. Particularly preferred agents contain at least one aminofunctional silicone of formula (Si-IIa)—

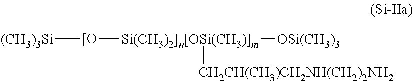

wherein m and n are numbers whose sum (m+n) is equal to a value from 1 to 2000, preferably from 50 to 150, n assuming values preferably from 0 to 1999 and particularly from 49 to 149, and m assuming values preferably from 1 to 2000, particularly from 1 to 10. These silicones are referred to according to the INCI declaration as Trimethylsilylamodimethicones.

Also particularly preferred are agents containing an aminofunctional silicone of formula (Si-IIb)—

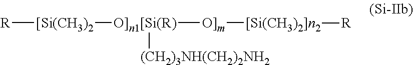

wherein R is —OH, —O—CH₃ or a —CH₃ group, and m, n1, and n2 are numbers whose sum (m+n1

+n2) is equal to a value from 1 to 2000, preferably from 50 to 150, the sum (n1+n2) assuming values preferably from 0 to 1999, particularly from 49 to 149, and m assuming values preferably from 1 to 2000, particularly from 1 to 10. These silicones are referred to according to the INCI declaration as Amodimethicones.

Regardless of which aminofunctional silicones are used, agents according to the present invention that contain an aminofunctional silicone whose amine number is above 0.25 meq/g, preferably above 0.3 meq/g, and in particular above 0.4 meq/g, are preferred. The amine number represents, in this context, milliequivalent amine per gram of aminofunctional silicone. It can be ascertained by titration, and also indicated using the unit of “mg KOH/g”. Preferred agents contain, based on their weight, 0.01 to 10 wt, by preference 0.1 to 8 wt %, particularly preferably 0.25 to 7.5 wt %, and in particular 0.5 to 5 wt % aminofunctional silicone(s).

“Clear” products are often preferred by consumers for aesthetic reasons. Hair treatment agents preferred according to the present invention are therefore transparent or translucent. “Transparent or translucent” is understood in the context of the present invention as a composition having an NTU value below 100. The NTU (nephelometric turbidity unit) is a unit used in water treatment for turbidity measurements in liquids. It is the unit of a liquid's turbidity, measured with a calibrated nephelometer.

Furthermore, in a preferred embodiment of the invention, an agent according to the present invention can also contain UV filters (I).

Agents according to the present invention can furthermore contain a 2-pyrrolidone-5-carboxylic acid and/or derivative thereof (J). Sodium, potassium, calcium, magnesium, or ammonium salts wherein the ammonium ion carries one to three C₁ to C₄ alkyl groups in addition to hydrogen are preferred. The sodium salt is very particularly preferred. The quantities used in agents according to the invention are preferably 0.05 to 10 wt %, particularly preferably 0.1 to 5, and in particular 0.1 to 3 wt %, based on total weight of the agent.

In addition, it may prove advantageous if penetration adjuvants and/or swelling agents (M) are present in the agents. These include, for example, urea and urea derivatives, guanidine and derivatives thereof, arginine and derivatives thereof, water glass, imidazole and derivatives thereof, histidine and derivatives thereof, benzyl alcohol, glycerol, glycol and glycol ethers, propylene glycol and propylene glycol ethers, (e.g., propylene glycol monoethyl ether), carbonates, hydrogencarbonates, diols and triols, and in particular 1,2-diols and 1,3-diols (e.g., 1,2-propanediol, 1,2-pentanediol, 1,2-hexanediol, 1,2-dodecanediol, 1,3-propanediol, 1,6-hexanediol, 1,5-pentanediol, 1,4-butanediol).

Advantageously for purposes of the invention, short-chain carboxylic acids (N) can additionally assist the active-substance complex (A). “Short-chain carboxylic acids” and derivatives thereof for purposes of the invention refer to carboxylic acids that can be saturated or unsaturated and/or straight-chain or branched or cyclic and/or aromatic and/or heterocyclic, and have a molecular weight below 750. Saturated or unsaturated straight-chain or branched carboxylic acids having a chain length from 1 to 16 carbon atoms in the chain can be preferred, with those having a chain length from 1 to 12 carbon atoms being particularly preferred.

Short-chain carboxylic acids include, for purposes of the invention, one, two, three, or more carboxy groups. Carboxylic acids having multiple carboxy groups, particularly di- and tricarboxylic acids, are preferred for purposes of the invention. The carboxy groups can be present entirely or partly as an ester, acid anhydride, lactone, amide, imidic acid, lactam, lactim, dicarboximide, carbohydrazide, hydrazone, hydroxam, hydroxim, amidine, amidoxime, nitrile, phosphonic ester, or phosphate ester. Carboxylic acids used according to the present invention can be substituted along the carbon chain or ring structure. Substituents of carboxylic acids used according to the present invention include C1 to C8 alkyl, C2 to C8 alkenyl, aryl, aralkyl, and aralkenyl, hydroxymethyl, C2 to C8 hydroxyalkyl, C2 to C8 hydroxyalkenyl, aminomethyl, C2 to C8 aminoalkyl, cyano, formyl, oxo, thioxo, hydroxy, mercapto, amino, carboxy, or imino groups. Preferred substituents are C1 to C8 alkyl, hydroxymethyl, hydroxy, amino, and carboxy groups. Substituents in the D position are particularly preferred. Very particularly preferred substituents are hydroxy, alkoxy, and amino groups, in which context the amino function can optionally be further substituted with alkyl, aryl, aralkyl, and/or alkenyl residues. In addition, phosphonic esters and phosphate esters are likewise preferred carboxylic acid derivatives.

In a further preferred embodiment, agents according to the present invention can contain emulsifiers (F). Emulsifiers cause the formation, at the phase interface, of water- or oil-stable adsorption layers that prevent the dispersed droplets from coalescing, and thereby stabilize the emulsion. Emulsifiers are therefore, like surfactants, constructed from a hydrophobic and a hydrophilic molecule part. Hydrophilic emulsifiers preferentially form oil-in-water (“o/w”) emulsions, and hydrophobic emulsifiers preferentially form water-in-oil (“w/o”) emulsions. An “emulsion” refers to a droplet-like distribution (dispersion) of one liquid in another liquid, with the expenditure of energy to create stabilizing phase interfaces by means of surfactants. Selection of these emulsifying surfactants or emulsifiers is based on the substances to be dispersed and the respective external phase, and on the fineness of the emulsion particles. Useful emulsifiers include—
addition products of 4 to 30 mol ethylene oxide and/or 0 to 5 mol propylene oxide with linear fatty alcohols having 8 to 22 carbon atoms, with fatty acids having 12 to 22 carbon atoms, and with alkylphenols having 8 to 15 carbon atoms in the alkyl group;
C₁₂ to C₂₂ fatty acid mono- and diesters of addition products of 1 to 30 mol ethylene oxide with polyols having 3 to 6 carbon atoms, in particular with glycerol;
addition products of ethylene oxide and polyglycerol with methyl glucoside fatty acid esters, fatty acid alkanolamides, and fatty acid glucamides;
C₈ to C₂₂ alkyl mono- and oligoglycosides and ethoxylated analogs thereof, degrees of oligomerization from 1.1 to 5, in particular 1.2 to 2.0, and glucose as the sugar component, being preferred;
mixtures of alkyl(oligo)glucosides and fatty alcohols, for example, the commercially available product Montanov® 68;
addition products of 5 to 60 mol ethylene oxide with castor oil and hardened castor oil;
partial esters of polyols having 3 to 6 carbon atoms with saturated fatty acids having 8 to 22 carbon atoms;
Sterols. “Sterols” are understood as a group of steroids that carry a hydroxyl group on the third carbon atom of the steroid structure and are isolated both from animal tissue (zoosterols) and from vegetable fats (phytosterols). Examples of zoosterols are cholesterol and lanosterol. Examples of suitable phytosterols are ergosterol, stigmasterol, and sitosterol. Sterols called “mycosterols” are also isolated from fungi and yeasts;
Phospholipids. These are understood as principally the glucose phospholipids, which are obtained e.g. as lecithins or phosphatidylcholines from, for example, egg yolk or plant seeds (e.g., soybeans);
fatty acid esters of sugars and sugar alcohols such as sorbitol;
polyglycerols and polyglycerol derivatives such as polyglycerol poly-12-hydroxystearate (commercial product Dehymuls® PGPH);
linear and branched fatty acids having 8 to 30 carbon atoms, and the Na, K, ammonium, Ca, Mg, and Zn salts thereof.

Agents according to the present invention preferably contain emulsifiers in amounts of from 0.1 to 25 wt %, particularly 0.5 to 15 wt %, based on total agent.

Compositions according to the present invention can preferably contain at least one nonionogenic emulsifier having an HLB value from 8 to 18. Nonionogenic emulsifiers having an HLB value from 10 to 15 can be particularly preferred according to the present invention.

It is additionally advantageous if, in addition to cationic and/or amphoteric polymer(s), further polymers (G) are present in the agents. In a preferred embodiment, further polymers are therefore added to the agents, with both anionic and nonionic polymers being effective.

Anionic polymers (G2) include anionic polymers having carboxylate and/or sulfonate groups. Examples of such anionic monomers are acrylic acid, methacrylic acid, crotonic acid, maleic acid anhydride, and 2-acrylamido-2-methylpropanesulfonic acid. The acid groups can be present entirely or partially as a sodium, potassium, ammonium, mono- or triethanolammonium salt. Preferred monomers are 2-acrylamido-2-methylpropanesulfonic acid and acrylic acid.

Anionic polymers containing 2-acrylamido-2-methylpropanesulfonic acid as a sole monomer or co-monomer have proven to be very particularly effective, wherein the sulfonic acid group can be present entirely or partially as a sodium, potassium, ammonium, mono- or triethanolammonium salt.

The 2-acrylamido-2-methylpropanesulfonic acid homopolymer that is available commercially, for example, under the tradename Rheothik® 11-80, is particularly preferred.

Within this embodiment, it may be preferred to use copolymers of at least one anionic monomer and at least one nonionogenic monomer. With regard to the anionic monomers, reference is made to the substances listed above. Preferred nonionogenic monomers include acrylamide, methacrylamide, acrylic acid ester, methacrylic acid ester, vinylpyrrolidone, vinyl ether, and vinyl ester.

Preferred anionic copolymers are acrylic acid/acrylamide copolymers, particularly polyacrylamide copolymers with sulfonic acid group-containing monomers. A particularly preferred anionic copolymer is made up of 70 to 55 mol % acrylamide and 30 to 45 mol % 2-acrylamido-2-methylpropanesulfonic acid, the sulfonic acid group being present entirely or partially as a sodium, potassium, ammonium, mono-, or triethanolammonium salt. This copolymer can also be present in crosslinked form, with polyolefinically unsaturated compounds such as tetraallyoxyethane, allylsucrose, allylpentaerythritol, and methylene bisacrylamide preferably being used as crosslinking agents. One such polymer is contained in the commercial product Sepigel® 305 of the SEPPIC Company. Utilization of this compound, which in addition to the polymer component contains a hydrocarbon mixture (C₁₃ to C₁₄ isoparaffin) and a nonionogenic emulsifier (Laureth-7), has proven particularly advantageous in the context of the teaching according to the present invention.

Sodium acryloyl dimethyl taurate copolymers marketed under the tradename Simulgel® 600 as a compound with isohexadecane and polysorbate-80 have also proven particularly effective according to the present invention.

Similarly preferred anionic homopolymers are uncrosslinked and crosslinked polyacrylic acids. Allyl ethers of pentaerythritol, of sucrose, and of propylene can be preferred crosslinking agents. Such compounds are obtainable commercially, for example, under the trademark Carbopol®.

Copolymers of maleic acid anhydride and methylvinyl ether, particularly those having crosslinks, are also color-preserving polymers. A maleic acid/methylvinyl ether copolymer crosslinked with 1,9-decadiene is commercially available under the tradename Stabileze® QM.

In a further embodiment, agents according to the present invention can contain nonionogenic polymers (G4).

Suitable nonionogenic polymers include vinylpyrrolidone/vinyl ester copolymers, cellulose ethers such as hydroxypropyl cellulose, hydroxyethyl cellulose, and methylhydroxypropyl cellulose, starch and derivatives thereof, particularly starch ethers, shellac, polyvinylpyrrolidone, siloxanes, glycosidically substituted silicones.

It is also possible for the preparations to contain multiple, in particular two, different polymers of the same charge, and/or respectively one ionic and one amphoteric and/or nonionic polymer.

Further polymers (G) can be present in the agents, preferably in quantities from 0.05 to 10 wt %, based on total agent. Quantities from 0.1 to 5, in particular from 0.1 to 3 wt %, are particularly preferred.

A further subject of the present invention is the use of fruit juices and/or vegetable juices in hair treatment agents in order to improve at least one of the following properties:
fullness and/or volume of keratinic fibers, in particular human hair, and/or
combability, in particular wet combability, of keratinic fibers, in particular human hair, and/or
tensile strength of keratinic fibers, in particular human hair, and/or
stabilization of the moisture budget of keratinic fibers, in particular human hair, and/or
combability of keratinic fibers, in particular human hair, and/or
delaying the aging process of keratinic fibers, in particular human hair, and/or
restructurability of keratinic fibers, in particular human hair, during and after the permanent-wave process, and/or
reducing the decrease in the elasticity of keratinic fibers, in particular human hair, when damaged by atmospheric effects.

With regard to preferred embodiments of the uses according to the present invention, the statements made with regard to the agents according to the present invention apply mutatis mutandis.

We claim:

1. Hair treatment agent comprising, in a cosmetically acceptable carrier:
0.1 to 35 wt % of at least one surfactant, and
15 to 85 wt % of at least one fruit juice or vegetable juice, wt % based on total weight of the agent.

2. Hair treatment agent according to claim 1, wherein the at least one fruit juice or vegetable juice is at least one or more fruit juices present in an amount of 20 to 80 wt %, based on total weight of the agent.

3. Hair treatment agent according to claim 1, wherein the at least one fruit juice or vegetable juice is at least one or more vegetable juices present in an amount of 20 to 80 wt %, based on total weight of the agent.

4. Hair treatment agent according to claim 1, wherein the at least one fruit juice or vegetable juice is at least one or more fruit juices chosen from apple juice, orange juice, grape juice, lemon juice, lime juice, sweet lime juice, grapefruit juice, blackcurrant juice, cherry juice, morello cherry juice, apricot juice, peach juice, nectarine juice, damson juice, yellow plum juice, passion fruit juice, plum juice, greengage juice, blackberry juice, raspberry juice, strawberry juice, pineapple juice, pomegranate juice, lychee juice, mango juice, tangerine juice, papaya juice, pitaya juice, carambola juice, pear juice, quince juice, loquat juice, serviceberry juice, rowan berry juice, gooseberry juice, blueberry juice, foxberry juice, tayberry juice, cranberry juice, elderberry juice, chokeberry juice, sea buckthorn berry juice and mixtures thereof.

5. Hair treatment agent according to claim 1, wherein the at least one fruit juice or vegetable juice is at least one or more vegetable juices chosen from rhubarb juice, kohlrabi juice, celeriac juice, carrot juice, beet juice, onion juice, leek juice, garlic juice, cabbage juice, asparagus juice, spinach juice, Swiss chard juice, tomato juice, cucumber juice, pumpkin juice, pea juice, bean juice, artichoke juice, cauliflower juice, kale juice, red cabbage juice, broccoli juice, Brussels sprout juice and mixtures thereof.

6. Hair treatment agent according to claim 1, wherein the at least one fruit juice or vegetable juice is an organic fruit juice or vegetable juice.

7. Hair treatment agent according to claim 1, wherein the agent is substantially free of compounds comprising the following formula (V) group—

8. Hair treatment agent according to claim 1, wherein the at least one surfactant is at least one or more amphoteric surfactants chosen from N-alkylglycines, N-alkylpropionic acids, N-alkylaminobutyric acids, N-alkyliminodipropionic acids, N-hydroxyethyl-N-alkylamidopropylglycines, N-alkyltaurines, N-alkylsarcosines, 2-alkylaminopropionic acids having approximately 8 to 24 carbon atoms in the alkyl group, alkylaminoacetic acids having approximately 8 to 24 carbon atoms in the alkyl group, N-cocoalkylaminopropionate, cocoacylaminoethylaminopropionate, C₁₂- to C₁₈-acyl sarcosine, N-alkyl-N,N-dimethylammonium glycinates, N-acylaminopropyl-N,N-dimethylammonium glycinates, 2-alkyl-3-carboxymethyl-3-hydroxyethylimidazolines having 8 to 18 carbon atoms in the alkyl or acyl group, cocoacylaminoethylhydroxyethylcarboxymethyl glycinate, compounds known by the INCI name Cocoamidopropyl Betaine, and/or compounds known by the INCI name Disodium Cocoamphodiacetate; and
wherein the one or more amphoteric surfactants are present in an amount of 0.5 to 9 wt %, based on total weight of the agent.

9. Hair treatment agent according to claim 1, wherein the at least one surfactant is at least an betaine amphoteric surfactant according to the following formula (VI)— 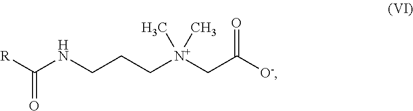
wherein R is a straight-chain or branched, saturated or mono- or polyunsaturated alkyl or alkenyl residue having 8 to 24 carbon atoms.

10. Hair treatment agent according to claim 9, wherein the residue R in formula (VI) is H₃C—(CH₂)₇—, H₃C—(CH₂)₉—, H₃C—(CH₂)₁₁—, H₃C—(CH₂)₁₃—, H₃C—(CH₂)₁₅—, H₃C—(CH₂)₇—CH═CH—(CH₂)₇—, or mixtures thereof.

11. Hair treatment agent according to claim 1, wherein the at least one surfactant is at least an betaine amphoteric surfactant according to the following formula (VII)— 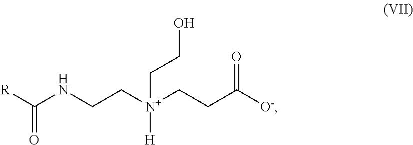
wherein R is a straight-chain or branched, saturated or mono- or polyunsaturated alkyl or alkenyl residue having 8 to 24 carbon atoms.

12. Hair treatment agent according to claim 10, wherein the residue R in formula (VII) is H₃C—(CH₂)₇—, H₃C—(CH₂)₉—, H₃C—(CH₂)₁₁—, H₃C—(CH₂)₁₃—, H₃C—(CH₂)₁₅—, H₃C—(CH₂)₇—CH═CH—(CH₂)₇—, or mixtures thereof.

13. Hair treatment agent according to claim 1, wherein the at least one surfactant is at least one or more alkylpolyglycoside nonionic surfactants of the general formula RO—(Z)_{X}, wherein R is alkyl, Z sugar, and x the number of sugar units, wherein the one or more alkylpolyglycoside nonionic surfactants are present in an amount of 0.1 to 20 wt %, based on total weight of the agent.

14. Hair treatment agent according to claim 1 further comprising 0.1 to 20 wt % fatty acid glutamates (acyl glutamates) and/or fatty acid aspartates (acyl aspartates) and/or alkylsulfoacetates (sulfoacetic acid alkyl esters), based on total weight of the agent.

15. Hair treatment agent according to claim 1 further comprising 0.001 to 10 wt % care-providing substances chosen from L-carnitine and/or salts thereof, panthenol and/or pantothenic acid, 2-furanones and/or derivatives thereof, taurine and/or salts thereof, niacinamide, ubiquinone, ectoin, and/or allantoin.
